(19) United States
(12) Patent Application Publication
(10) Pub. No.: US 2019/0076634 A1
(43) Pub. Date: Mar. 14, 2019

(54) STERILIZING AND NUMBING KIT

(71) Applicant: Charmaine McHugh, Ft. Lauderdale, FL (US)

(72) Inventor: Charmaine McHugh, Ft. Lauderdale, FL (US)

(21) Appl. No.: 15/703,337

(22) Filed: Sep. 13, 2017

(51) Int. Cl. A61F 13/40; A61K 31/167; A61K 31/245; A61L 2/00; A61M 19/00; A61M 35/00; B65D 81/22

(52) U.S. Cl. CPC A61M 35/006; A61K 31/167; A61K 31/245; B65D 81/22; A61M 19/00; A61M 35/003; A61L 2/0088

(57) ABSTRACT

A sterilizing and numbing kit for sterilizing and numbing a topical area including a swab. The swab includes a grip and a fluid absorbable material attached to the grip. The fluid absorbable material is saturated with a sterilizing agent and a topical anesthetic. A sealed cover is provided being positioned over and sealing the fluid absorbable material.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to sterilizing and numbing agents and more particularly pertains to a new topical sterilizing and numbing kit for easy application of sterilizing and numbing agents to a topical area.

### BRIEF SUMMARY OF THE INVENTION

An embodiment of the disclosure meets the needs presented above by generally comprising a sterilizing and numbing kit including a swab. The swab includes a grip and a fluid absorbable material attached to the grip. The fluid absorbable material is saturated with a sterilizing agent and a topical anesthetic. A sealed cover is provided being positioned over and sealing the fluid absorbable material.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a sterilizing and numbing kit according to an embodiment of the disclosure.

FIG. 2 is a top side view of an embodiment of the disclosure.

FIG. 3 is a cross-sectional view of an embodiment of the disclosure taken along the line 3-3 of FIG. 2.

### DETAILED DESCRIPTION OF THE INVENTION

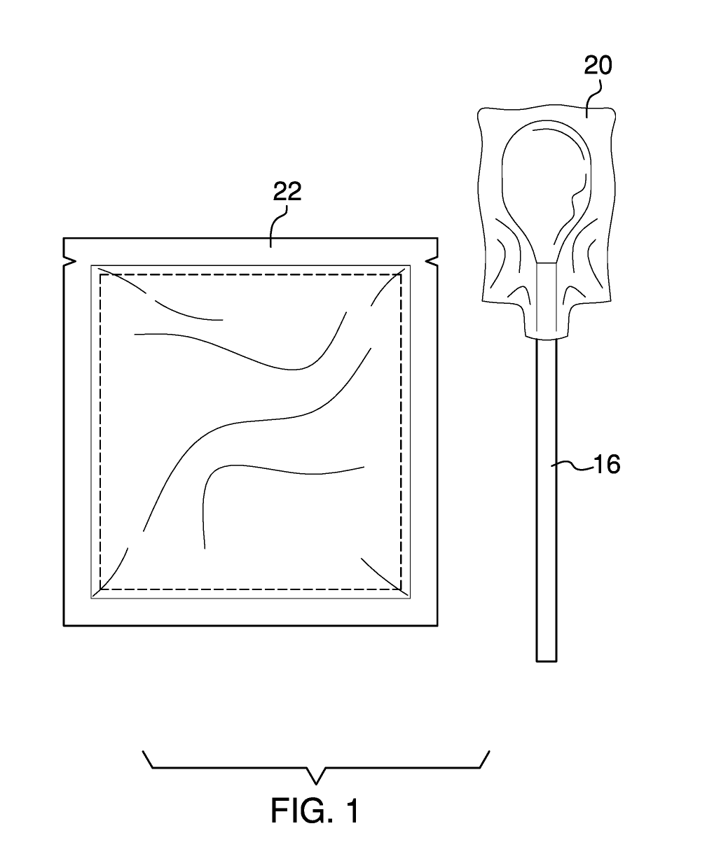

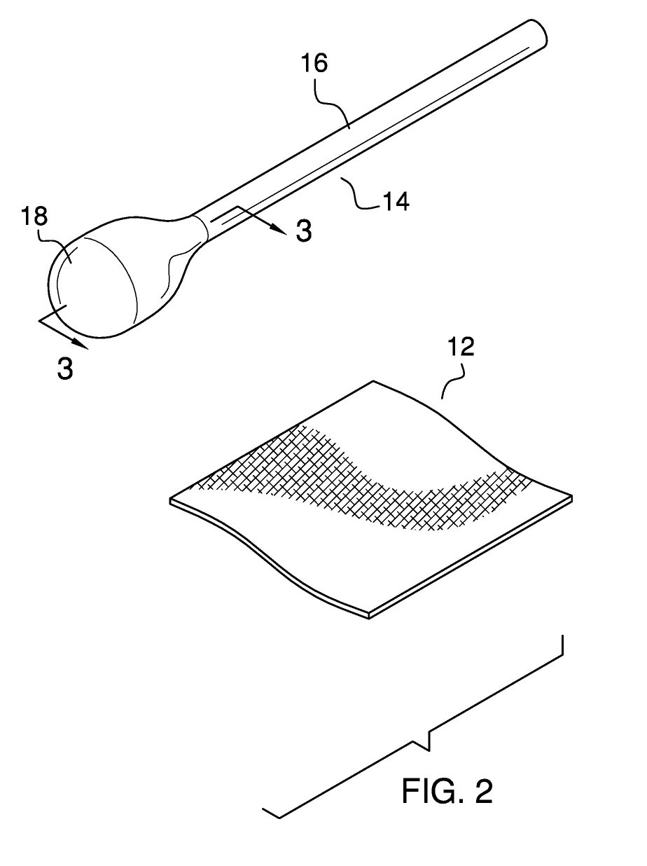

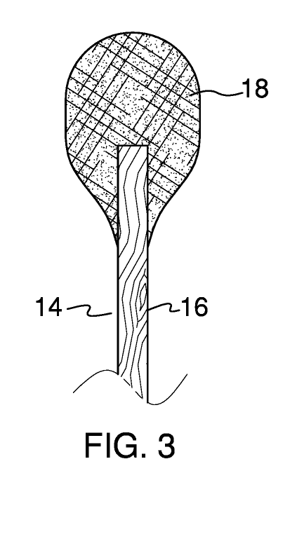

With reference now to the drawings, and in particular to FIGS. 1 through 3 thereof, a new sterilizing and numbing kit embodying the principles and concepts of an embodiment of the disclosure and generally designated by the reference numeral 10 will be described.

As best illustrated in FIGS. 1 through 3, the sterilizing and numbing kit 10 generally comprises a towelette 12 and a swab 14. The swab 14 includes a grip 16 and a fluid absorbable material 18 attached to the grip 16. The fluid absorbable material 18 should be saturated with both a sterilizing agent and a topical anesthetic. A sealed cover 20 is positioned over and seals the fluid absorbable material 18 within the cover.

The grip 16 may be elongated wood. It should be understood that the grip can be other suitable shapes and other suitable material for directing the fluid absorbable material 18 such as rolled paper or plastic. The fluid absorbable material 18 may be wrapped around the grip 16, or attached by other methods. The fluid absorbable material 18 is most often a cotton material, but may comprise other material capable of retaining fluids. The cover 20 should contain at least the whole of the fluid absorbable material 18 portion of the swab as to contain the sterilizing agent and topical anesthetic. However, the cover 20 may contain the grip 16 and any part thereof. The cover may be any material suitable to prevent the sterilizing agent and topical anesthetic from leaking out of the cover 20 and therefore may comprise a plastic or sealed paper material.

A towelette 12 may also be provided. It may be made of any material suitable for absorbing fluid such as cotton materials provided in various shapes and sizes. The towelette 12 should be saturated with a sterilizing agent and a topical anesthetic. The towelette 12 is contained within a sealed pouch 22 constructed to contain the saturated towelette 12 without allowing the sterilizing agent or topical anesthetic to leak outwardly therefrom.

While one of multiple sterilizing agents may be utilized, one appropriate sterilizing agent is isopropyl alcohol. The topical anesthetic may be particularly selected from one of the group including lidocaine, novocain or benzocaine. Lidocaine is also known as xylocaine or lignocaine and novocain is also known as procaine.

In use, the kit 10 is used as a quick and effective means to sterilize and anesthetize a topical area. The kit 10 provides the option between a towelette 12 and a swab 14, both with their own advantages. Both the towelette 12 and the swab 14 may be conventionally used to disperse the sterilizing agent and the topical anesthetic over a topical area. The towelette 12 may sterilize and anesthetize a larger surface area, while the swab 14 may allow for more targeted dispersion of the sterilizing agent and the anesthetic. When a person is to receive a shot or have blood drawn, the kit 10 allows for simultaneous sterilizing and anesthetizing for patient comfort.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sterilizing and numbing kit comprising:
a swab including a grip and a fluid absorbable material attached to said grip, said fluid absorbable material being saturated with a sterilizing agent and a topical anesthetic;
a sealed cover positioned over and sealing said fluid absorbable material.

2. The sterilizing and numbing kit of claim 1, said sterilizing agent comprising isopropyl alcohol.

3. The sterilizing and numbing kit of claim 1, said topical anesthetic being selected from the group including lidocaine, novocain or benzocaine.

4. The sterilizing and numbing kit of claim 1, said sterilizing agent comprising of isopropyl alcohol, said topical anesthetic being selected from the group including lidocaine, novocain or benzocaine.

5. The sterilizing and numbing kit of claim 1, further comprising a towelette saturated with a sterilizing agent and a topical anesthetic.

6. The sterilizing and numbing kit of claim 5, further comprising a sealed pouch, said towelette being positioned within said sealed pouch.

7. A sterilizing and numbing kit comprising:
a swab including a grip and a fluid absorbable material attached to said grip, said fluid absorbable material being saturated with a sterilizing agent and a topical anesthetic, said sterilizing agent comprising isopropyl alcohol, said topical anesthetic being selected from the group including lidocaine, novocain or benzocaine;
a sealed cover positioned over and sealing said fluid absorbable material;
a towelette saturated with a sterilizing agent and a topical anesthetic, said sterilizing agent comprising isopropyl alcohol, said topical anesthetic being selected from the group including lidocaine, novocain or benzocaine; and
a sealed pouch, said towelette being positioned within said sealed pouch.
